(19) United States
(12) Patent Application Publication
(10) Pub. No.: US 2006/0229244 A1
(43) Pub. Date: Oct. 12, 2006

(54) Engineered bacteriocins and bacteriocin combinations and methods for treating bacterial based infections

(76) Inventors: Robert Dorit, Leeds, MA (US); Margaret A. Riley, Northampton, MA (US)

Correspondence Address: Clark Hill PLC, Suite 3500, 500 Woodward Avenue, Detroit, MI, 48226-3435, US

(21) Appl. No.: 10/953,615

(22) Filed: Sep. 27, 2004

(51) Int. Cl. A61K 38/16

(52) U.S. Cl. 514/12

(57) ABSTRACT

A method for treating bacterial infections in a patient is provided. The method includes the step of administering a therapeutically effective amount of a single naturally-occurring or engineered bacteriocin, or combinations thereof, designed to have high specific activity against the bacterial infection to the patient so that a rate of resistance to the bacteriocin in the patient is decreased. The colicins and other bacteriocins or combinations of colicins and other bacteriocins target and kill specific bacterial pathogens in a manner that results in a high specific killing activity and decreased incidence of pathogen resistance. To this end, the characteristics of existing colicins and other bacteriocins are modified in order to enhance and amplify their therapeutic value.

### BACKGROUND OF THE INVENTION

1. Field of the Invention

The invention relates to a method for treating bacterial infections. More particularly, the invention relates to a method of treating bacterial infections in patients using a naturally occurring or engineered bacteriocin, or combinations thereof.

2. Description of the Related Art

Antibiotics are generally defined as substances that are derived from bacterial sources for killing the bacteria that cause infections. Bacteriocins are substances produced by certain bacteria for killing or inhibiting the growth of other closely related bacterial strains. Thus, bacteriocins are natural antibiotics. Naturally occurring bacteriocins already exist to treat all known human pathogens. Most human and animal pathogens exhibit sensitivity to one or more existing bacteriocins. One class of bacteriocins, known as colicins, have been shown to kill uropathogenic and diarrheagenic strains of E. coli, including serotype O157:H7 and its derivatives, currently the most significant diarrhea-producing strains of E. coli. More recent work has illustrated that colicins are effective against many additional pathogenic enteric bacteria, including Salmonella typhimurium, Klebsiella pneumoniae, Enterobacter cloacae, and Hafnia alvae.

Colicins exhibit many properties sought in any potential therapeutic antimicrobial. It is, however, appreciated that all bacteriocins produced by gram-Negative bacteria share similar properties. Colicins are a class of high molecular weight protein antimicrobials that are secreted into the environment by producer E. coli strains, and act to inhibit or kill sensitive conspecifics (E. coli) or related bacteria (principally members of the Enterobacteriaceae) through mechanisms including pore formation, inhibition of cell wall synthesis, DNA degradation, and RNA cleavage. The well-characterized kinetics of colicin activity approximate single-hit dynamics, suggesting that the entry of a single colicin molecule into a sensitive cell is sufficient to result in target cell death.

Colicins recognize receptors on the target cell surface, including the BtuB and FepA cell surface receptors. These receptor systems—indispensable cell-surface receptors evolved to carry out important metabolic functions in E. coli—are co-opted by the colicins to gain entry into the target cell. Following binding to the receptor, the colicin is translocated through the membrane using transport systems such as the Tol and TonB systems.

The structure of colicins makes these proteins ideal candidates for in vitro engineering. Structural studies reveal that the majority of colicins is composed of several stable, independently-folding domains connected to on another by compact and stable hinge regions. The stability conferred by this arrangement is further complemented by the structural and functional modularity of the colicin domains. A variety of colicin domain constructs, which incorporate only parts of the colicin gene, have been shown to adopt the proper structural configuration, suggesting the existence of only a limited number of critical inter-domain contacts.

The applied potential of bacteriocins has already been demonstrated. The bacteriocin nisin has been used in a variety of applied settings. Nisin is an effective inhibitor of Erwinia, Pseudomonas and Xanthomonas growth on vegetables and other foodstuffs, and of Listeria monocytogenes on smoked fish and milk. Nisin has also been used to inhibit plaque-producing bacteria, and appears to strongly inhibit the growth of a variety of multi-drug resistant gram-positive pathogens, including S. aureus, S. pneumoniae, and E. faecalis. Nisin has been recognized as safe in the United States for use in selected pasteurized cheese spreads to prevent spore outgrowth and toxin production by C. botulinum, as a preservative to extend shelf life of dairy products, and in spoilage prevention in canned goods.

A lozenge containing bacteriocin-producing Streptococcus is available in New Zealand for the treatment of throat infections. And a mouse model has been established showing that colicins are highly effective in the mouse colon at removing targeted E. coli strains (Kirkup and Riley, submitted).

In recent years, it has been noticed that antibiotics have become less effective as patients use them more frequently. This is due to the fact that bacterial pathogens build up a resistance to the antibiotic over time. The therapeutic history of antibiotics suggests that for every novel antibiotic drug designed or discovered, it is almost always the case that the microbial community already harbors at least a partial solution to the task of antibiotic resistance. Thus, the problem of antibiotic resistance in bacterial pathogens continues to increase and now presents a significant challenge, both within and outside the hospital environment.

The use of multi-component therapies, that is, the use of multiple antibiotics together in the same dose, has generated significant interest for many decades. Multi-component combinations promise two significant therapeutic benefits: 1) a significant reduction (often below detectable levels) of the overall pathogen load in treated patients, resulting in improved clinical outcomes; and 2) decreased appearance of pathogen isolates resistant to the multi-component therapy, even in light of the very high mutation rate characteristic of microbial pathogens.

Despite these advantages, the utility of multi-component therapies has, up to now, been severely limited by the difficulty and cost involved in generating each member of a multi-component set. The current strategy for antimicrobial discovery has no built-in economies of scale, and is seldom geared to the identification of multiple related antimicrobials. Instead, current strategies of drug discovery focus on single highly active compounds, which are independently isolated and refined. Only after several of such individual compounds have been fully and independently developed are they, on occasion, administered as multi-component combinations. Under existing approaches, the timeline for development of a single antibiotic runs to 10 years (or more) at a cost ranging from tens to hundreds of millions of dollars.

A rapid method for the simultaneous development and identification of multiple, related, active antimicrobials is needed. It would be desirable for such a method to include built-in economies of scale to remove one of the main obstacles, that is, cost, limiting the greater use of multi-component therapies. To this end, the activity of engineered bacteriocins was examined to identify multiple, related active compounds. While the exact relationship among these active compounds cannot be predicted a priori, they provide a continuous and inexpensive input of candidate leads for the exploration of the behavior and characteristics of multi-component antimicrobial therapies.

### BRIEF SUMMARY OF THE INVENTION

According to one embodiment of the invention, a method for treating bacterial infections in a patient is provided. The method includes the step of administering a therapeutically effective amount of a single, naturally-occurring or engineered bacteriocin, or combinations thereof, specifically designed to have high specific activity against the bacterial infection to the patient so as to reduce or eliminate the infection while simultaneously ensuring that the rate of resistance to the bacteriocin or bacteriocin combination in the patient is decreased.

### BRIEF DESCRIPTION OF THE DRAWINGS

Advantages of the present invention will be readily appreciated as the same becomes better understood by reference to the following detailed description when considered in connection with the accompanying drawings wherein:

FIG. 1 depicts the three-dimensional structure inferred for a colicin E9 cassette III variant as created in Protein Explorer and includes receptor recognition 10, translocation 20, and killing domains 30 in the protein structure;

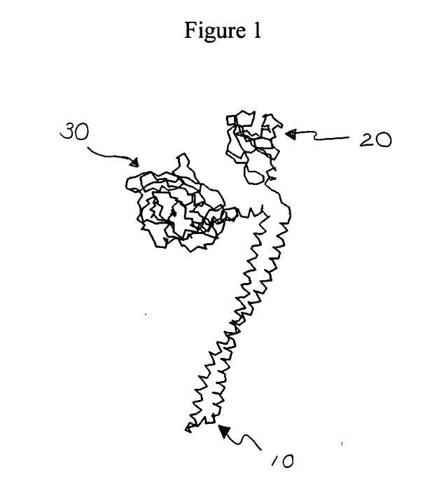

FIG. 2 illustrates the enteric bacteriocin phylogenetic killing range with the frequency of bacteriocin killing within each of seven enteric taxa mapped onto a composite molecular phylogeny of enteric bacteria wherein the bacteriocins assayed for killing breadth are indicated across the top (with the corresponding abbreviations displayed at the branch tips) and each column provides the frequency of killing for each bacteriocin assayed against ˜40 indicator strains for each taxa in the molecular phylogeny;

FIG. 3a illustrates a minimum lethal dose (MLD) as determined upon exposing 10⁸ sensitive BZB1011 cells to increasing amounts of colicin K lysate wherein the number of surviving cells decreases until the MLD is reached, at which point only resistant cells are seen;

FIG. 3b illustrates the effect of colicin lysates, administered singly, pairwise, and in three-way combinations wherein the log of the absolute value of the resistance frequency is plotted for the individual components of the combination (e.g. colicins E3, K or A) as well as for the pairwise and three-way combinations;

FIG. 4 illustrates the effect of colicin combinations on resistance frequency of sensitive E. coli with the first three bars in each set depicting resistance frequencies to individual colicins and the fourth (darker) bar depicting resistance frequencies to three-way combinations of colicins wherein the data are plotted as the log of the absolute value of the observed resistance frequency;

FIG. 5 is a schematic diagram of the plasmid created for colicin cassette (Cassette 1 of 3) mutagenesis wherein the variously hashed regions of the plasmid indicate the distinct functional domains of the colicin E9 protein encoded and the two expanded portions reveal the amino acid sequence of the target region and the sequence of the degenerate oligomer constructed for mutagenesis;

FIG. 6 illustrates the minimum lethal dose (MLD) for engineered colicin C1-33 as determined upon exposing 10⁸ sensitive BZB1011 cells to increasing amounts of colicin C1-33 lysate wherein the number of surviving cells decreases until the MLD is reached, at which point only resistant cells are seen (in this example at 15-20 ul of lysate); and

FIG. 7 is a schematic of a strategy for the creation of novel proprietary hybrid bacteriocin molecules depicting the generation of proprietary hybrid DNA sequences encoding novel bacteriocin proteins, wherein the DNA sequences are subsequently expressed in an inducible expression system and screened for antibiotic activity, and detailing the isolation and purification strategies resulting in active bacteriocin molecules.

### DETAILED DESCRIPTION OF THE PREFERRED EMBODIMENTS

According to one embodiment of the invention, a method for treating a bacterial infection in a patient includes the step of administering a therapeutically effective amount of a single, naturally-occurring or engineered bacteriocin, or combinations thereof, designed to have high specific activity against the bacterial infection to the patient so that the infection is resolved and the rate of resistance to the bacteriocin compound or composition in the patient is decreased. In one preferred embodiment of the invention, the engineered bacteriocins are the product of PCR-mediated or ligation-mediated recombination, as well as site-directed, cassette-mediated mutagenesis, or other methods for the randomization of particular positions or domains of the bacteriocin molecule followed by selection for high specific activity of a combination of functional domains and subsequent sequence modification from naturally occurring bacteriocins of any Gram-negative bacteria. It is, however, appreciated that the engineered bacteriocins may be a product of PCR-mediated recombination and selection for high specific activity of a combination of functional domains and subsequent sequence modification from naturally occurring bacteriocins of any eubacteria, including Gram-positive bacteria, and archaebacteria. In another preferred embodiment of the invention, the engineered bacteriocins are the product of PCR-mediated recombination and selection for high specific activity of a combination of functional domains and subsequent sequence modification from one or more of the following naturally occurring pyocins: S1, S2, S3, S5, AP41, C. And in still another preferred embodiment of the invention, the engineered bacteriocins are the product of PCR-mediated recombination and selection for high specific activity of a combination of functional domains and subsequent sequence modification from one or more of the following naturally occurring colicins—A, B, D, DF13, E1-E9, EL12, G, H, Ia, Ib, K, L, M, N, S1, S4, U, Y, 5, 7, 10, 28b, Hu194, and J.

In yet another preferred embodiment of the invention, a combination of engineered bacteriocins is utilized for treating the bacterial infection wherein each of the engineered bacteriocins are the product of PCR-mediated recombination and selection for high specific activity of a combination of functional domains and subsequent sequence modification from naturally occurring bacteriocins of any gram-Negative bacteria, as well as other eubacteria, and archaebacteria.

In another preferred embodiment of the invention, a combination of naturally occurring bacteriocins is utilized for treating the bacterial infection. In one preferred embodiment, the combination of naturally occurring bacteriocins is naturally occurring colicins. In another preferred embodiment, the combination of naturally occurring bacteriocins is a combination of naturally occurring eubacteria or archaebacteria.

The term “therapeutically effective amount” used herein refers to that amount of the bacteriocin that is sufficient for treating, as defined below, a bacterial infection in a patient when administered thereto. The therapeutically effective amount will vary depending upon the subject and disease condition being treated, the weight and age of the patient, the severity of the disease condition, and the manner of administration and the like, which can be readily determined by one of ordinary skill in the art.

The term “treating” used herein means any treatment of a disease in a patient including: (1) preventing the disease, that is, causing the clinical symptoms of the disease not to develop; (2) inhibiting the disease, that is, arresting the development of clinical symptoms; and/or (3) relieving the disease, that is, causing the regression of clinical symptoms.

The term “patient” includes mammals and non-mammals. The mammals include humans and non-human animals.

The engineered bacteriocins are chosen based upon high specific activity against any human or animal pathogen. Such high specific activity is attained in the engineered colicins and other bacteriocins through altered receptor recognition, translocation, and killing domains. The reliance of colicins on receptors and translocation systems that play an integral role in the survival of the cell makes deletion of receptors or translocation proteins an unlikely resistance strategy for the target cell, particularly in non-laboratory environments.

The activity of the colicin—receptor-binding, translocation and killing activity—can be localized to discrete, collinear segments of the colicin gene. This modular gene architecture suggests that individual aspects of the phenotype, such as the interaction of the colicin protein with the receptor, the translocation of the colicin into the cytoplasm of the target cell, and the killing of the target cell, can be independently manipulated. Consequently, the interaction between colicin and the receptor and the subsequent translocation of colicin can be altered and enhanced in our studies without disrupting the killing activity of the modified colicin molecules.

The in vivo role of colicins requires these proteins to be effectively exported into the extra-cellular medium. This is accomplished through the activity of a lysis protein, which is co-transcribed with the colicin protein. When colicin production is induced, the lysis protein disrupts the cell membrane of the producing cell, causing the release of the cytoplasmic contents, which may include over 30% colicin protein. Thus, the engineered colicins are able to be readily released into the extra-cellular medium, where their anti-microbial characteristics can be fully harnessed.

In one preferred embodiment of the invention, the engineered bacteriocins are chosen based upon high specific activity against uropathogenic E. coli and other enteric pathogens. In another preferred embodiment of the invention, the engineered bacteriocins are chosen based upon high specific activity against pathogenic strains of Salmonella typhimurium and other enteric pathogens. In yet another preferred embodiment of the invention, the engineered bacteriocins are chosen based upon high specific activity against pathogenic Pseudomonas aeruginosa.

The therapeutically effective amount of the engineered bacteriocin is provided for treating or preventing various bacterial infections in patients, particularly humans and other animals. The method can be applied to all bacterial infections, regardless of their resistance status. These bacterial infections include, but are not limited to, urinary tract infections, urogenital infections, gastrointestinal infections, skin infections, respiratory infections in mammals. The therapeutically effective amount of the engineered bacteriocin may be delivered to the patient using a pharmaceutically acceptable carrier. Although this invention is not intended to be limited to any particular mode of application, it is preferred that the mode of application for the therapeutically effective amount be oral, intravaginal, intraurethral, or periurethral. More particularly, the therapeutically effective amount of the bacteriocin may be installed in the form or a pill, injectable patch, injectable syringe, cream, liquid, paste, gel, or suppository as desired. One preferred form is a cream formulation including one or more bacteriocin combinations in a jelly base, preferably a K-jelly base.

The term “pharmaceutically acceptable carrier” used herein means one or more compatible solid or liquid filler diluents, or encapsulating substances. By “compatible” as used herein is meant that the components of the composition are capable of being commingled without interacting in a manner which would substantially decrease the pharmaceutical efficacy of the total composition under ordinary use situations.

Some examples of substances that can serve as pharmaceutical carriers are sugars, such as lactose, glucose, and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations. Wetting agents and lubricants such as sodium lauryl sulfate, as well as colouring agents, flavouring agents, lubricants, excipients, tabletting agents, stabilizers, anti-oxidants and preservatives, can also be present.

One group of patients at risk of acquiring a urinary tract infection are those requiring long term and intermittent catheterization. Catheterization causes trauma and acts as a focus for pathogenic bacteria to colonize the uroepithelium and the catheter itself in dense microcolonies, which are resistant to antibiotic penetration, leading to persistent infection. It is possible to coat the uroepithelium and catheter surfaces with bacteriocin combinations thereby excluding pathogens from colonizing and causing an infection. Accordingly, in a further aspect of this invention, a novel method of treating or preventing urinary tract infections is provided which involves coating or otherwise incorporating one or more of these proprietary molecules into a biologically compatible prosthetic device for subsequent use or insertion in surgical or therapeutic interventions (urogenital tract). The biologically compatible prosthetic device may be composed of polymers such as fluorinated ethylene propylene, sulfonated polystyrene, polystyrene, or polyethylene terephthalate and in addition, other plastics, composites or glass. The device may be a catheter such as a urinary or peritoneal catheter or other intravaginal, intrauterine, or intraurethral device.

Not only would males and females in need of a treatment for urinary tract infection benefit from this method, but also females not immediately in need of such treatment but who can be considered “prone” to urinary tract infections would benefit from this invention. These individuals can benefit by treatment using one or more bacteriocin combinations to reduce or eliminate potential enteric pathogens in their colon or vaginal region. In addition, this invention has potential applications as a preventative measure to reduce the probability of acquiring a urinary tract infection, or other bacterial infection.

The ability of bacteriocin combinations to target specific uropathogenic E. coli is influenced by numerous factors and effects including the cell surface receptor and translocation system of the pathogen. Although the invention is not bound by any one theory or mode of operation, it is believed that, at least to some degree, a combination of engineered bacteriocins in a combination form may be responsible for excluding pathogens and reducing their numbers in the urinary tract.

The in vitro approach described herein emphasizes the rapid creation and isolation of large families of active bacteriocin compounds. When coupled with the reduced resistibility of combination antimicrobial therapies, the methods herein represent a powerful, practical, and statistically significant strategy for the discovery of new antibiotics. As a result, a significant arsenal of compounds of potential therapeutic utility in the treatment of human and animal bacterial infections is possible.

The invention will now be illustrated by means of the following non-limiting examples.

### EXAMPLE 1

### Partial Purification of Colicin Proteins and Their Activity Against an Indicator Strain

One ml of a fresh overnight growth of each wild-type colicin producer cell line is transferred into 50 ml fresh LB media in 250 mL Erlenmeyer flasks. The cultures were grown at 37° C. with agitation for approximately 90 minutes. When the A₆₀₀ reached OD of >0.2, 500 μl of a 50 μg/ml solution of Mitomycin C are added (final concentration: 0.05 μg/ml) to induce colicin production, and growth is continued for three to six hours. The cells are lysed by adding 3 ml of chloroform and vortexing. After centrifugation at 10,000 g for 10 minutes, the supernatant containing the colicin molecules is transferred to a clean tube and partially purified and concentrated using Centricon Plus-20 spin columns (P1-30, 30,000 NMWL) according to manufacturer's instructions, generally resulting in a 100-500-fold concentration of the colicin molecules. The resulting concentrates can be used directly, or stored at −20° C. Fifty μl of a fresh overnight growth of sensitive E. coli BZB1011 is added to 4 ml of top agar (20 g/L LB, 7 g/L Bacto-Agar) and poured onto an LB plate. Two μl of lysate of each wild-type colicin is then spotted twice onto this BZB1011 lawn. After overnight incubation at 37° C., the phenotype is scored as (+++) when the colicin produces a clear zone of growth inhibition, as (++) when the colicin produces a visible translucent clearing zone of reduced diameter (relative to wt), and as (+) when it produced a visible, but faint clearing in the lawn with an opaque plaque. When no zone of inhibition is seen, the phenotype is scored as (−).

### EXAMPLE 2

### Colicin Activity Against Uropathogenic E. coli

Using the methods described in Example 1, the sensitivity of strains of uropathogenic E. coli to naturally occurring colicins was assayed. The uropathogenic E. coli are a sample of clinical isolates obtained from women presenting with cystitis and related urinary tract infections. Table 1 shows the sensitivity of uropathogenic E. coli to naturally occurring colicins. This study established that colicins are effective antimicrobials against uropathenogenic E. coli, underscoring the potential of colicins in the treatment and prevention of urinary tract infections.

TABLE 1

| UTI Strain | Colicin Sensitivity |
|---|---|
| 36 | E1, E3, E7, E9, A, D, K, Ia, Ib, M, N, C1.9, C2.11 |
| 45 | A, D, K |
| 177 | E1, A, D, K, Ib |
| 457 | E1, E3, E7, E9, A, D, K, Ib, C1.9, C2.11 |
| 458 | E1, E3, E7, E9, A, D, K, Ia, Ib, N, C1.9, C2.11 |
| 473 | E1, A, D, K |

### EXAMPLE 3

### Bacteriocin Activity Against Enteric Bacteria

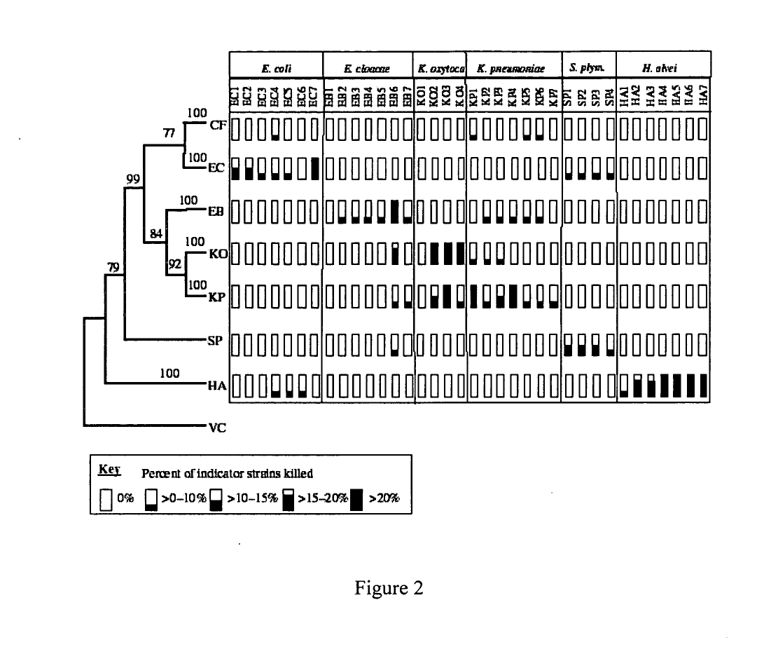

A phylogenetic-based screen was performed to reveal the specificity of naturally occurring bacteriocins. A well-characterized collection of over 500 strains of enteric bacteria isolated from wild mammals in Australia was assayed for bacteriocin production and sensitivity. FIG. 2 summarizes these data. The results most relevant to this study include: (i) bacteriocins are found at high levels in natural populations of enteric bacteria, (ii) these naturally occurring toxins are most effective at killing members of the same species, (iii) certain bacteriocins show high levels of activity against other species of bacteria, and (iv) there are numerous bacteriocins with strain- and species-specific activity. The work is detailed in Wertz et al., 2003.

### EXAMPLE 4

### Bacteriocins Combinations Retain Activity Against Sensitive Bacteria

Combinations of naturally occurring colicins were prepared and assayed against enteric bacteria. The general protocol in these experiments consisted of: 1) establishing the minimum lethal dose of a given colicin extract that will result in the death of all sensitive cells (given that approximately 10⁸ cells are plated in a given experiment); 2) combining two or more colicins at the minimum lethal dose; 3) comparing the lethality of the multicomponent combinations against that of a two- or three-fold dose of each single component in the combination. These steps are described in detail below.

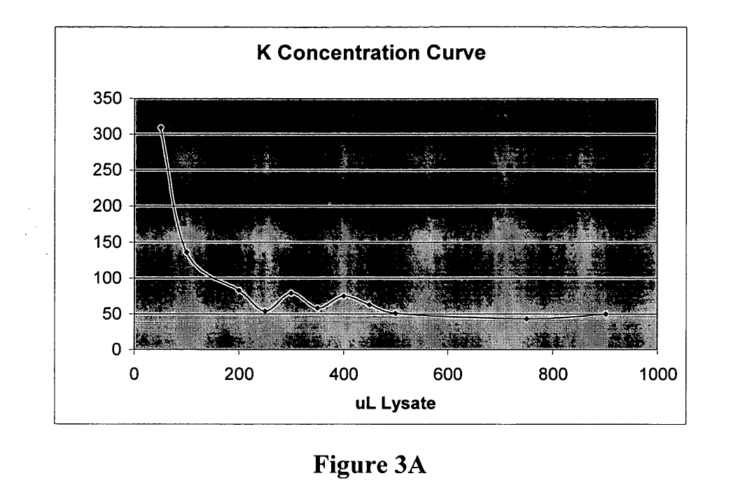

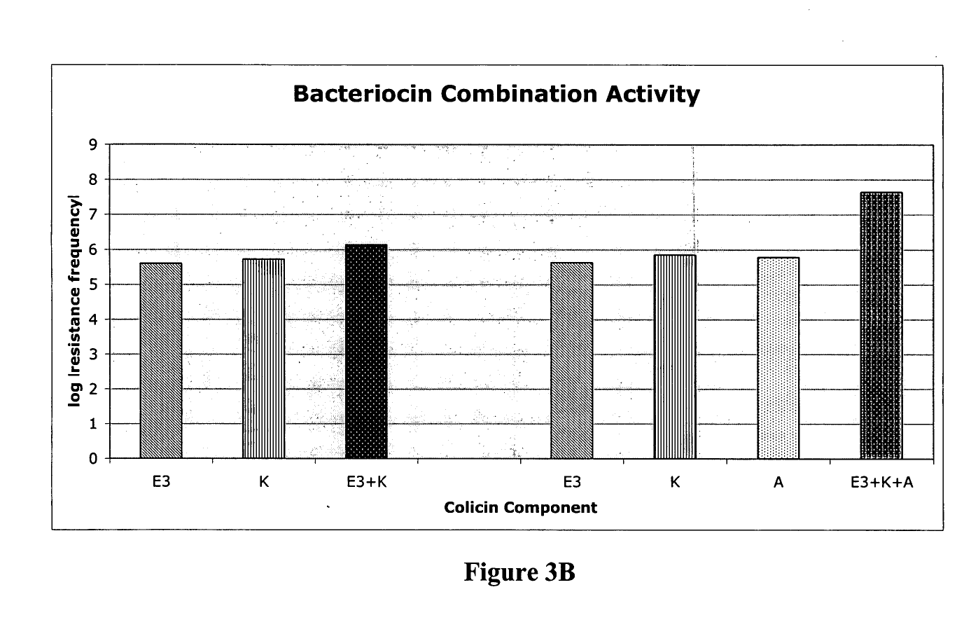

1) Minimum lethal dose: An example of this assay, using colicin K, is shown in FIG. 3a, where 100 ul of BZB1011 sensitive cells (containing approximately 10⁸ cells) are mixed with varying amounts of lysate prepared as described above, ranging, in the illustrated example, from 50 to 900 ul. The mixture of cells and lysate is vortexed and immediately spread onto LB plates. The plates are then incubated overnight at 37° C. and the surviving colonies (resistant to the lysate) counted. The dose of lysate that results in the survival of only resistant cells, visible as the point in the curve where the number of surviving cells no longer varies with lysate amount is chosen as the minimum lethal dose (200 ul of lysate in the example shown).

2) Creating combinations: Once the minimum lethal dose has been established for a number of colicins, these are combined into a mixture containing two or three different bacteriocins, and >10⁸ sensitive cells added and plated as described above. The plates are incubated overnight and the number of surviving colonies counted and recorded.

3) Scoring killing activity. The bactericidal activity of the combinations is compared to that of the individual components, and the results recorded and plotted as the log of the absolute value of the resistant frequency. An example of these comparisons, for a two and a three-component combination, are shown in FIG. 3b. As can be seen, combinations exhibit high specific activity against enteric bacteria.

### EXAMPLE 5

### Bacteriocin Combinations Result in a Significant Decrease in the Frequency of Resistance

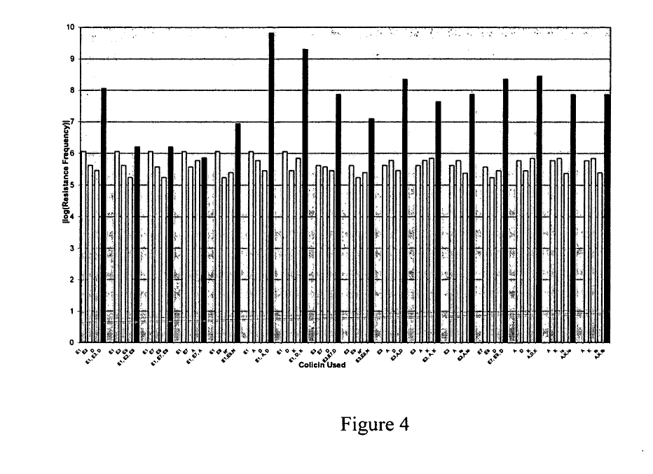

FIG. 4 provides a comparison of the levels of colicin resistance observed when colicins are administered singly or in combinations for a variety of colicins. The methods employed in this study are detailed in part in Example 4. Resistance is assayed by counting surviving colonies following exposure to lethal concentrations of colicin as previously determined, see Example 4. This study establishes that the use of colicin combinations results in high, specific activity and results in a decrease in the rate of colicin resistance evolution by as much as 4 orders of magnitude (1×10⁻¹¹ mutations per cell per generation).

### EXAMPLE 6

### The Design of Novel, High Activity Engineered Colicin Molecules

Cell lines: The host producer cell line E. coli JM83 [F-_(lac-proAB) phi80_(lacZ)M15 ara rpsL thi lambda-] was obtained from the American Type Culture Collection. The cell line used for plasmid construction, E. coli DH5∝[supE44 lacU169 (lacZM15) hsd R17 recA1 endA1 gyrA96 thi-1 re1A1] was obtained from Gibco BRL. E. coli BZB1011, used in all assays as the colicin-sensitive indicator strain (“target cell”) was obtained from Dr. A. P. Pugsley and has previously been described. All cultures were routinely grown under standard conditions in Luria-Bertani (LB) broth or on LB agar plates supplemented when required with ampicillin (50 μg/ml).

Design of the mutagenic cassette: In this study we have focused on a 10 AA cassette (AA 423 to 432) located at the C-terminal region of the R domain of colicin E9. Rather than allow for full degeneracy of the 30 nt. cassette encoding the region of interest, we opted instead for a mutagenesis design that favored the overall conservation of the polar/nonpolar character of the AA in this domain. The synthetic oligomer containing the degenerate cassette also included conserved 5′ (44 nt) and 3′ (33 nt) flanking regions that allowed for the amplification, restriction digestion and cloning of the mutagenized cassette.

The mutagenic oligodeoxynucleotide cassette MCI was chemically synthesized (1 umol scale). The oligodeoxynucleotide, AAT TTA TCC TTG GCG TCC TTT TCT TTA TTT TCC TTC TGT TTG CG VAV 5TS 5KS 5TS 5TS 5TS 5KS VAV VAV VAV AGC ATC TGA CTT CTC TTT TGC GGC CGC ATC AAA, contained partially randomized positions 2014 to 2043 of the 5523 nt long pMc27 sequence (see below and FIG. 2) (5=3:3:3:1 proportion for A:C:G:T, V=1:1:1 proportion for A:C:G, S=proportion for C:G, K=proportion for T:G; underlined sequences=constant primer-binding sequences; StyI and EagI restriction sites are italicized). This mutagenic oligonucleotide was subsequently made into a double-stranded fragment with PCR amplification using flanking oligomers, complementary to the conserved regions. This PCR was performed in six reaction mixtures (100 ul each) containing 4 nmol of each primer (MCA.fwd, 5′-AAT TTA TCC TTG GCG TCC-3′ and MCB.rev,5′-TTT GAT GCG GCC GCA AAA G-3′), 3.8 ug of ssDNA template, PCR buffer (10× contains Tris-HCI, KCI, (NH₄)₂SO₄, 15 mM MgCl₂; pH 8.7), and 50 U of HotStart Taq DNA polymerase (QIAGEN, Germany) with a GeneAmp thermocycler (Model 9700; Perkin Elmer), under the following amplification conditions: ten cycles of amplification; initial denaturation at 94° C. for 15 min, annealing at 60° C. for 1 min, primer extension at 72° C. for 1 min, denaturation at 94° C. for 1 min. and final extension at 72° C. for 7 min. The resulting amplified DNA was purified using the PCR Purification Kit according to manufacturer's instructions (QIAGEN, Germany) and double-digested with the restriction endonucleases EagI and StyI (1.8 ug DNA, 30 U each enzyme from NEB, 2 h at 37° C.).

Plasmid construction: The initial plasmid used in this study, pMC27, was kindly provided by Dr. C. Penfold and has previously been described. The plasmid consists of a pUC 18 backbone into which the colicin gene cluster containing the entire colicin E9 coding sequence (ceaI), as well as the E9 immunity gene (ceiI) and lysis gene (lys) have been inserted. In order to permit the rapid cloning of the mutagenized cassette, the pMC27 plasmid was modified by the addition of two new restriction sites. This was accomplished by identifying sites that differ at a single nucleotide from canonical unique restriction sites.

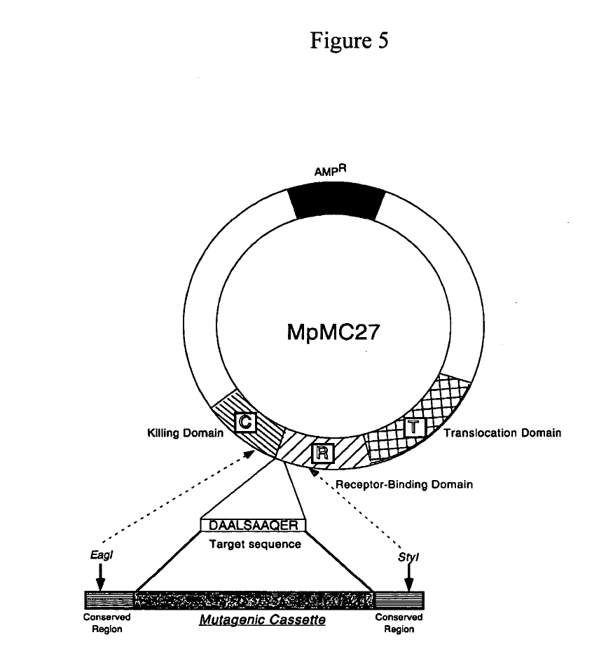

Plasmid pMC27 pMC27 was isolated from E. coli JM83 using the QIAprep Miniprep Kit (QIAGEN, Germany). Fifty nanograms of plasmid DNA was amplified by PCR using 130 ng of each mutagenic primer (StyI.fwd, 5′-AGA AAA GGA CGC CAA GGA TAA ATT-3′ and StyI.rev, 5′-AAT TTA TCC TTG GCG TCC TTT TCT-3′), synthesis buffer (10× buffer contains 200 mM Tris-HCl, 100 mM KCl, 100 mM (NH₄)₂SO₄, 20 mM MgSO₄, 1.0% Triton X-100 and 1 mg/ml BSA, pH 8.8 at 25° C.), and 4.5 U of Pfu DNA polymerase (Promega) with a GeneAmp thermocycler (Model 9700; Perkin Elmer). Fifteen cycles of amplification were employed, with initial denaturation of the DNA at 94° C. for 30 sec, annealing at 55° C. for 1 min, primer extension at 68° C. for 12 min. and denaturation at 94° C. for 30 sec. The parental methylated DNA template was digested with DpnI endonuclease (10 U enzyme from NEB, 1 h at 37° C. The nicked vector DNA incorporating the desired mutation was then transformed into E. coli DH5 supercompetent cells (Life Technologies) following the protocol recommended by the manufacturer. Plasmid DNA from the transformed cells was then isolated using the Q1Aprep Miniprep Kit (QIAGEN, Germany) and digested with the restriction endonuclease StyI to confirm the desired mutation. The DNA was then used as the template in a second site-directed mutagenesis performed as described above with the following modification: mutagenic primers EagI.fwd, 5′-GCA TTT GAT GCG GCC GCA AAA GAG AAG-3′ and EagI.rev, 5′-CTT CTC TTT TGC GGC CGC ATC AAA TGC-3′. PCR was performed using 55° C. for 1 min. for annealing. The desired mutation was confirmed by digestion of the plasmid vector with the restriction endonuclease EagI. This resulted in the creation of unique sites in the plasmid that did not alter the coding sequence of the colicin E9 ceaI gene. The sequence of the modified plasmid (MpMc27) was subsequently verified by sequencing; the overall organization of the plasmid is shown in FIG. 5.

MpMC 27 was then restriction digested with EagI and StyI, creating a directional cloning orientation and compatible ends for the insertion of the mutagenic cassette. The plasmid was dephosphorylated using Calf Intestinal Alkaline Phosphatase according to manufacturer's instructions. Dephosphorylated plasmid was purified from agarose gel using the Gel Purification Kit (QIAGEN, Germany). The mutagenic cassette was subsequently ligated using T4 DNA with Ligase according to manufacturer's instructions.

Transformation and Screening: MpMC27 plasmids containing variant colicin plasmids were transformed via electroporation into JM83 cells (50 ul volume of cells, 0.1 cm cuvette gap, 1.8 kV Voltage, 18 kV/cm field strength, 25 uF capacitor, 200 Ω resistance, 4.2-5.0 msec time constant; Gene Pulser II, Bio-Rad ), resulting in transformation efficiencies of approximately 6×10⁷ CFU/ug DNA (1.5×10⁹ cells/ml). Our original protocol called for the simultaneous screening of lysates derived from 10⁷ cells, each likely to contain a different variant of colicin E9 (for the mutagenized 10 AA region of the R domain). It soon became clear, however, that this could be replaced by a simpler assay involving replica plating of transformed JM83 cells containing the variant MpMC27 plasmids, first onto LB agar containing ampicillin and subsequently onto LB plates pre-seeded with a lawn of sensitive E. coli. BZB1011, using sterile velvet pads. The lawns were prepared by adding 10⁶ sensitive cells and 0.5 ug/ml Mitomycin C to 7 ml top agar (20 g/L LB, 7 g/L Bacto-Agar) and poured over LB plates. After overnight incubation at 37° C., the plates were inspected for the presence of a clearing diameter on the BZB1011 lawn, indicating an active variant. Alignment of the LB/Amp and BZB1011 lawn plates allowed individual colonies to be selected, retested for active colicin production and further characterized.

Sequence analysis: Colony PCR was performed using reaction mixtures (50 ul each) containing 10 pmol of each primer (PMC27A.rev, 5′-GCT CCT GAA TCT TTA CCT GC-3′ and PMC27B.fwd, 5′-GGT CAC AGA ATG TGG CAA ATG G-3′), PCR buffer (10× contains Tris-HCl, KCl, (NH₄)₂SO₄, 15 mM MgCl₂; pH 8.7), and 1.25 U of HotStart Taq DNA polymerase (QIAGEN, Germany). Each colony was picked with a sterile toothpick and transferred to the PCR master mix. Twenty-five cycles of amplification were employed, with initial denaturation of the DNA at 94° C. for 15 min., annealing at 60° C. for 1 min, primer extension at 72° C. for 1 min., denaturation at 94° C. for 1 min. and final extension at 72° C. for 7 min. Amplified DNA was purified using the PCR Purification Kit (QIAGEN, Germany).

A first screening of mutagenic clones was performed by digestion of an amplified product aliquot with the restriction endonuclease MseI (NEB, 2h at 37° C.), directed at a site present in the wild-type sequence, but unlikely to be conserved in any of the engineered variants. The products of digestion were analyzed on a 3% agarose gel, and clones exhibiting a band pattern different from the wild type sequence were selected for sequencing. DNA sequencing was performed using the BigDye Terminator Kit (Perkin Elmer) according to manufacturer's instructions, and products were visualized on the automated ABI 377 sequencer. This work was published in Dorit and Riley 2002. Subsequent cassette constructions include: cassette 2 and 3, which target additional regions of the colicin E9 receptor-binding domain (unpublished).

### EXAMPLE 7

Activity of engineered colicins and engineered colicin combinations against sensitive cells

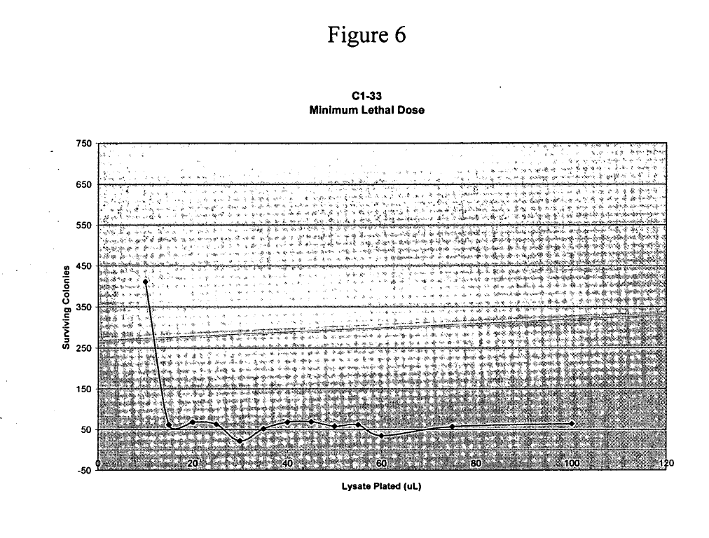

Engineered colicins, singly and in combination, were created that result in high activity against sensitive cells. An example of the activity and minimum lethal dose of one of our engineered constructs, C1-33, is shown in FIG. 6. The methods employed are as given above.

### EXAMPLE 8

### Designing Engineered Bacteriocins

Naturally occurring bacteriocins, along with their cognate immunity proteins, have been cloned into the Quiagen pQe vector system, which allows the purification via His-tagging of the expressed bacteriocin protein.

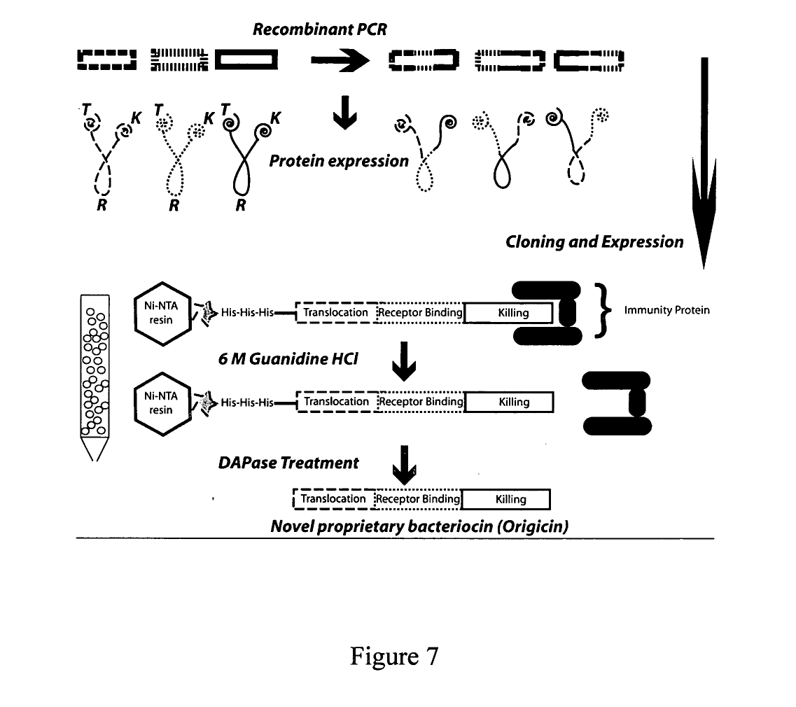

We have cloned a cassette containing the bacteriocin and a 6-His N-terminal tag, along with the immunity protein, under the control of the inducible promoter. The immunity protein is thus co-expressed with the bacteriocin, and allows for high levels of expression in the producer cell. Cells are then lysed, and the resulting protein lysates passed through Ni-purification columns, which selectively retain His-tagged proteins. The bacteriocin/immunity complex is thus retained in the column, which is subsequently washed with a 6M-guanidium chloride solution, releasing the immunity protein while still retaining the His-tagged bacteriocin molecule. After column equilibration, the bacteriocin is released via enzymatic cleavage using DAPase digestion (Tagzyme). As illustrated in FIG. 7, we have engineered the colicin backbone to include unique restriction sites at the domain boundaries, thus allowing us to explore the results of PCR-mediated recombination either within the translocation or receptor binding domains alone, or for the molecule as whole.

The novel molecules will be generated using a modified version of recombinant PCR (“sexual PCR”) methods. These approaches involve the generation of heterologous molecules as a result of repeated cycles of annealing and extension in the presence of heterogeneous templates. The PCR is always primed with a set of conserved flanking markers, allowing the subsequent cloning of the novel products. This approach has been successfully exploited to create a number of proteins with desired phenotypes, and to optimize the catalytic profile of existing enzymes. In addition, we created chimeric molecules by using a modified version of a heteroduplex recombination approach. In this strategy, mixtures of vectors harboring homologous (but non-identical) genes, each cut once at a unique restriction site are denatured and allowed to anneal, creating heteroduplex plasmids with a single-stranded break in each strand, which are subsequently targeted by the repair machinery of the transformed cell. The resulting products are novel combinations of sequences from the original donor sequences, and these can be further assayed for antibiotic activity.

The invention has been described in an illustrative manner. It is to be understood that the terminology, which has been used, is intended to be in the nature of words of description rather than of limitation. Many modifications and variations of the invention are possible in light of the above teachings. Therefore, within the scope of the appended claims, the invention may be practiced other than as specifically described.

1. A method for treating a bacterial infection in a patient, the method comprising the step of administering a therapeutically effective amount of an engineered bacteriocin designed to have high specific activity against the bacterial infection to the patient so that the bacterial infection is suppressed or cured and the rate of resistance to the engineered bacteriocin in the patient is decreased.

2. A method as set forth in claim 1 wherein the bacteriocin is a bacteriocin produced by any Gram-negative bacteria.

3. A method as set forth in claim 2 wherein the bacteriocin is produced from the group of colicins consisting of: A, B, D, DF13, E1-E9, EL12, G, H, Ia, Ib, K, L, M, N, S1, S4, U, Y, 5, 7, 10, 28b, Hu194, and J.

4. A method as set forth in claim 2 wherein the bacteriocin is produced from a pyocin selected from the group of pyocins consisting of: S1, S2, S3, S5, AP41, and C.

5. A method as set forth in claim 1 wherein the bacteriocin is produced from a eubacteria.

6. A method as set forth in claim 1 wherein the bacteriocin is produced from an archaebacteria.

7. A method as set forth in claim 2 wherein the bacteriocin is a combination of bacteriocins produced by any Gram-negative bacteria.

8. A method as set forth in claim 1 including the step of delivering the therapeutically effective amount of the bacteriocin using a pharmaceutically acceptable carrier.

9. A method as set forth in claim 6 wherein the pharmaceutically acceptable carrier is selected from the group consisting of: a pill, an injectable patch, a cream, a liquid, a paste, a gel, and a suppository.

10. A method as set forth in claim 7 wherein the cream includes a jelly base.

11. A method as set forth in claim 1 including the step of coating at least a portion of a catheter with the therapeutically effective amount of the bacteriocin.

12. A method as set forth in claim 1 including the step of incorporating the therapeutically effective amount of the bacteriocin into a biologically compatible prosthetic device.

13. A method as set forth in claim 1 wherein the bacterial infection is a gastrointestinal infection.

14. A method as set forth in claim 1 wherein the bacterial infection is a urogenital infection.

15. A method as set forth in claim 1 wherein the bacterial infection is a skin infection.

16. A method as set forth in claim 1 wherein the bacterial infection is a respiratory infection.

17. A method as set forth in claim 1 wherein the patient is a mammal.

18. A method as set forth in claim 1 wherein the bacteriocin has a high specific activity against pathogenic Salmonella typhimurium.

19. A method as set forth in claim 1 wherein the bacteriocin has a high specific activity against pathogenic Pseudomonas aeruginosa.

20. A method for treating a urinary tract infection in a patient, the method comprising the step of administering a therapeutically effective amount of an engineered bacteriocin designed to have specific activity against the urinary tract infection to the patient so that the urinary tract infection in the patient is suppressed or cured and the rate of resistance to the engineered bacteriocin in the patient is decreased.

21. A method as set forth in claim 20 wherein the bacteriocin is a bacteriocin produced by any Gram-negative bacteria.

22. A method as set forth in claim 21 wherein the bacteriocin is produced from a colicin selected from the group of colicins consisting of: A, B, D, DF13, E1-E9, EL12, G, H, Ia, Ib, K, L, M, N, S1, S4, U, Y, 5, 7, 10, 28b, Hu194, and J.

23. A method as set forth in claim 21 wherein the bacteriocin is produced from a pyocin selected from the group of pyocins consisting of: S1, S2, S3, S5, AP41, and C.

24. A method as set forth in claim 20 wherein the bacteriocin is produced from a eubacteria.

25. A method as set forth in claim 20 wherein the bacteriocin is produced from an archaebacteria.

26. A method as set forth in claim 21 wherein the bacteriocin is a combination of bacteriocins produced by any Gram-negative bacteria.

27. A method as set forth in claim 20 including the step of delivering the therapeutically effective amount of the bacteriocin using a pharmaceutically acceptable carrier.

28. A method as set forth in claim 27 wherein the carrier is selected from the group consisting of: a pill, an injectable patch, a cream, a liquid, a paste, a gel, and a suppository.

29. A method as set forth in claim 28 wherein the cream includes a jelly base.

30. A method as set forth in claim 20 wherein the bacteriocin has a high specific activity against uropathogenic E. coli.

31. A method as set forth in claim 20 including the step of coating at least a portion of a catheter with the therapeutically effective amount of the bacteriocin.

32. A method as set forth in claim 20 including the step of incorporating the therapeutically effective amount of the bacteriocin into a biologically compatible prosthetic device.

33. A method as set forth in claim 20 wherein the patient is a mammal.

34. A method for treating a bacterial infection in a patient, the method comprising the step of administering a therapeutically effective amount of a combination of naturally occurring bacteriocins each having specific activity against the bacterial infection to the patient so that the bacterial infection is suppressed or cured and the rate of resistance to the combination of naturally occurring bacteriocins in the patient is decreased.

35. A method as set forth in claim 34 wherein the combination of bacteriocins is produced from a combination of naturally occurring colicins.

36. A method as set forth in claim 34 wherein the combination of bacteriocins is produced from a combination of naturally occurring eubacteria.

38. A method as set forth in claim 34 wherein the combination of bacteriocins is produced from a combination of naturally occurring archaebacteria.

39. A method as set forth in claim 34 including the step of delivering the therapeutically effective amount of bacteriocin to the patient using a pharmaceutically acceptable carrier.

40. A method as set forth in claim 34 including the step of coating at least a portion of a catheter with the therapeutically effective amount of the bacteriocin.

41. A method as set forth in claim 34 including the step of incorporating the therapeutically effective amount of the bacteriocin into a biologically compatible prosthetic device.


SEQUENCE LISTING (52 sequences)

SEQ ID NO: 1 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctggtctttgcccatcatgacgaggactttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 2 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtgctgttccatgatcagcaacaggaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 3 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtggttgttccgcagcatcaacccgacgtgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 4 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttcctccttgatgaggaccatgcacaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 5 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcttcctcgcccagcaccagccacatctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 6 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtcgtttttgcagaccaccatcagcaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 7 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttgttggttccccaagatgagccccacttccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 8 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcttcgtcccggatgaagatccacaggtccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 9 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctggtcttcccggaccagcaggaccagctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 10 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgcttttctcttgcctcaacacgacccggaagtccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 11 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtccttctcgcccatcatcaccagcacttgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 12 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgttcttcttcctcagcaggatgcccacctgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 13 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgttcttctccacgaccagcacgcacaggtccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 14 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctctttctcccggatgacgaggatcatctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 15 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttgtttctggccgatcaagatcctgggttccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 16 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttgctcgtcccggaccagcacgaacactaccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 17 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctgcttttccctcaacaccacgagcagtttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 18 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctgctgttcaacagcaccatgccgaaagttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 19 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcttcgtccacgaacagcacccccactttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 20 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcctcctggaccatcagcacgccgattttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 21 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcctgttgccacacgagcaagaggaggttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 22 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctccttctcccgcacgatgatgaacatctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 23 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttggtcgtccatcaggacgatgctgacctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 24 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctggtcttgcaccaccaccatgaacaagttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 25 - DNA, 1749 bp, Escherichia coli; identical to SEQ ID NO: 24

SEQ ID NO: 26 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctcttttgcttcatgacgacgaccccgatctgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 27 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttccttttggcccatcaagacgaccacctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 28 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctattcctggcccagcagcatcctcacctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 29 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctgttcgtccatgacgatgaccagcaacttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 30 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtcgttttgcaggaccagcacgcgcacttgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 31 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttcgtcctccatgatgaggacgatcatgttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 32 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgttcttttccaacaccaggatccgcacgttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 33 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctgctccttcaccatgagcatgacgacctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 34 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcctcgtccatcaacatcaacaggagttccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 35 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtcctgctgcatgatcacgaggccgacgtccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 36 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgcttttgtgctccaccacgaacatccggacttgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 37 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtcctcttccctgatcatgacgatgagcttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 38 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttcgttctcgccgaccatcaacaacacctccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 39 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtgctggtcgccgagcagcatgcccaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 40 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtggttctcccccacgatcaggaccaggttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 41 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtgctccttccgcatcatcacccgcattttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 42 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctctttttccctcaacacgacgcacattttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 43 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctcttctgctccaccatcatcacgctgatctgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 44 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttgctgttccacgaacacgacgaacacgttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 45 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttttttctccccgagcaccagcctcaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 46 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcctgcttcatgatcagcatccccagctgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 47 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctcgtgttgcatcatgaccacccccaatttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 48 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctctgctgctccctcaagaccacccagatgttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 49 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctcttcttgtgccccatcatcacgcggacgtccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 50 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgcttttttccttcagcacgatcatgaccagttccgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 51 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctttgtttttgcatgatcacgacgctgatctgcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa

SEQ ID NO: 52 - DNA, 1749 bp, Escherichia coli
atgagcggtggggatggacgcggccataacacgggcgcgcatagcacaagtggtaacattaatggtggcccgaccgggattggtgtaagtggtggtgcttctgatggttcaggatggagttcggaaaataacccgtggggtggtggttccggtagcggcattcactggggaggtggctccggtcgtggtaatggcgggggtaatggcaattccggtggtggctcgggaacaggcggtaatttgtcagcagtagctgcgccagtggcatttggttttccggctctttccactccaggagctggcggtctggctgtcagtatttctgcaagcgaattatcggcagctattgctggtattattgctaaattaaaaaaagtaaatcttaaattcactccttttggggttgtcttatcttcattaattccgtcggaaatagcgaaagatgaccccaatatgatgtcaaagattgtgacgtcattacccgcagatgatattactgaatcacctgtcagttcattacctctcgataaggcaacagtaaacgtaaatgttcgtgttgttgatgatgtaaaagacgaacgacagaatatttcggttgtttcaggtgttccgatgagtgttccggtggttgatgcaaaacctaccgaacgtccaggtgtttttacggcatcaattccaggtgcacctgttctgaatatttcagttaatgacagtacgccagcagtacagacattaagcccaggtgttacaaataatactgataaggatgttcgcccggcaggatttactcagggtggtaataccagggatgcagttattcgattcccgaaggacagcggtcataatgccgtatatgtttcagtgagtgatgttcttagtcctgaccaggtaaaacaacgtcaggatgaagaaaatcgccgtcagcaggaatgggatgctacgcatccggttgaagcggctgagcgaaattatgaacgcgcgcgtgcagagctgaatcaggcaaatgaagatgttgccagaaatcaggagcgacaggctaaagctgttcaggtttataattcgcgtaaaagcgaacttgatgcagcgaataaaactcttgctgatgcaatagctgaaataaaacaatttaatcgatttgcccatgacccaatggctggcggtcacagaatgtggcaaatggccgggcttaaagctcagcgggcgcagacggatgtaaataataagcaggctgcatttgatgctgctgcaaaagagaagtcagatgctgtgtttttggagcaccaccaggctcaccttcgcaaacagaaggaaaataaagaaaaggacgctaaggataaattagataaggagagtaaacggaataagccagggaaggcgacaggtaaaggtaaaccagttggtgataaatggctggatgatgcaggtaaagattcaggagcgccaattccagatcgcattgctgataagttgcgtgataaagaatttaaaagcttcgacgattttcggaaggctgtatgggaagaggtgtcgaaagatcctgagcttagtaaaaatttaaacccaagcaataagtctagtgtttcaaaaggttattctccgtttactccaaagaatcaacaggtcggagggagaaaagtctatgaacttcatcatgacaagccaattagtcaaggtggtgaggtttatgacatggataatatccgagtgactacacctaagcgacatatcgatattcaccgaggtaagtaa